(19) United States
(12) Patent Application Publication
(10) Pub. No.: US 2024/0318811 A1
(43) Pub. Date: Sep. 26, 2024

(54) Light Handle Cover

(71) Applicant: Daniel P. Saliaris, Delaware, OH (US)

(72) Inventor: Daniel P. Saliaris, Delaware, OH (US)

(21) Appl. No.: 18/614,946

(22) Filed: Mar. 25, 2024

Related U.S. Application Data
(60) Provisional application No. 63/454,168, filed on Mar. 23, 2023.

(51) Int. Cl. F21V 21/40; F21V 33/00; F21W 131/20

(52) U.S. Cl. CPC F21V 21/403; F21V 33/0068; F21W 2131/20

(57) ABSTRACT

A surgical light handle cover that covers the handle during surgical procedures to provide a sanitary environment. The cover is preferably made of an elastomeric material or at least a material that stretches when placed on the handle. The cover has a cylindrical portion that covers the handle and a conical portion that covers the base of the handle. An end portion opposite the conical portion covers the tip of the handle where some lights have switches that turn the light on and off when actuated. The cylindrical portion may be tapered with a larger diameter opening to facilitate insertion of the handle into the cylindrical portion, and the end portion may have a thinner sidewall than the cylindrical portion to ease switching of the light.

Patent application Ser. No. 63/454,168 filed Mar. 23, 2023, which is the claimed priority application, is incorporated in this application by reference.

### BACKGROUND OF THE INVENTION

The invention relates generally to sanitation in a surgical environment, and more specifically to a cover for the handle of an operating room light.

Modern operating rooms have common features, including lights above an operating table to enable the surgeon to better view the surgical site than using common light fixtures. A typical operating room's equipment is shown in FIG. 1 with two lights 2 and 3 disposed over the operating table 4. Enlarged images of two common lights used in operating rooms are illustrated in FIGS. 2 and 3. The lights shown have handles 7 at or near the center for a surgeon or an assistant to grasp, as shown taking place in FIG. 4, and then move the light to better illuminate the surgical site. The handles are conventionally about 4-5 inches long, with a base that is tapered and that attaches to, or is integral with, a shaft that is about one inch in diameter. This makes it very easy to grasp the handle and pivot the light using the human hand applying a force to the handle.

There are two types of surgical light handles that are common. One type has a button/switch 9 on the tip that allows the light to be adjusted by pressing the switch. The second type is similar but without a switch on the tip. The former type is shown in FIGS. 4 and 5, and the latter type is shown in FIGS. 2 and 3 and is switched at another location.

Because the handle 7 may be grasped during surgery, the handle must be covered with a removable cover. Furthermore, the cover must be replaced after each surgery. A conventional cover used on these handles consists of a vinyl sleeve that may be sold under the Mckesson brand and described as a “Light Handle Cover Flexible.” This cover is made of flexible plastic that has a generally cylindrical shape with a tapered base, and it assumes the shape of the light handle when installed. This type of cover is difficult to slide over the light handle. Furthermore, because the fit to the handle is a tight one, the installer must pull on it to get it in place. This type of cover is the consistency of plastic used to make thick, disposable garbage bags, which is not substantially elastic. Instead, this type of cover is thin enough to flex (bend) during installation, removal and use. There is a need for an improved light handle cover.

### SUMMARY OF THE INVENTION

Disclosed herein is a cover for a light handle. The cover comprises a substantially cylindrical portion having a barrel defined by a sidewall. The cover also comprises a conical portion disposed near a first end of the cylindrical portion. At least a portion of the conical portion has a larger diameter than the cylindrical portion. The cover also comprises an end portion disposed near a second end of the cylindrical portion opposite the conical portion.

In some embodiments, at least the substantially cylindrical portion and the end portion are made of an elastomeric material. In some embodiments, the substantially cylindrical portion is tapered so that the first end has a larger diameter than the second end. In some embodiments, a sidewall thickness of the cylindrical portion is larger than a sidewall thickness of the end portion.

Disclosed herein is a combination of a cover and a light handle. The combination comprises a substantially cylindrical portion having a barrel defined by a sidewall in which the light handle is disposed. The combination also comprises a conical portion disposed near a first end of the cylindrical portion over a base of the light handle. At least a portion of the conical portion has a larger diameter than the cylindrical portion. The combination also comprises an end portion disposed near a second end of the cylindrical portion opposite the conical portion and adjacent a light switch of the light handle.

In some embodiments, at least the substantially cylindrical portion and the end portion are made of an elastomeric material. In some embodiments, the substantially cylindrical portion is tapered so that the first end has a larger diameter than the second end. In some embodiments, a sidewall thickness of the cylindrical portion is larger than a sidewall thickness of the end portion.

### BRIEF DESCRIPTION OF DRAWINGS

FIG. 1 is a view in perspective illustrating prior art operating room equipment typical of a modern operating room.

FIG. 2 is a view in perspective illustrating a first prior art operating room light.

FIG. 3 is a view in perspective illustrating a second prior art operating room light.

FIG. 4 is a view in perspective illustrating a prior art operating room light with a person grasping the handle of the light to adjust it.

FIG. 5 is a view in perspective illustrating a conventional operating room light handle with a switch at the end.

FIG. 6 is a side view in section illustrating an embodiment of the present invention.

FIG. 7 is a side view in section illustrating the embodiment of FIG. 6.

FIG. 8 is an end view illustrating the embodiment of FIG. 6.

FIG. 9 is a view in perspective illustrating an embodiment of the invention in use.

FIG. 10 is a view in perspective illustrating the embodiment of FIG. 9 with a human finger approaching the end of the cover.

FIG. 11 is a view in perspective illustrating the embodiment of FIG. 9 with the human finger in contact with the end of the cover.

FIG. 12 is a view in perspective illustrating the embodiment of FIG. 9 with the human finger depressing the end of the cover.

In describing the preferred embodiment of the invention which is illustrated in the drawings, specific terminology will be resorted to for the sake of clarity. However, it is not intended that the invention be limited to the specific term so selected and it is to be understood that each specific term includes all technical equivalents which operate in a similar manner to accomplish a similar purpose. For example, the word connected or terms similar thereto are often used. They are not limited to direct connection, but include connection through other elements where such connection is recognized as being equivalent by those skilled in the art.

### DETAILED DESCRIPTION OF THE INVENTION

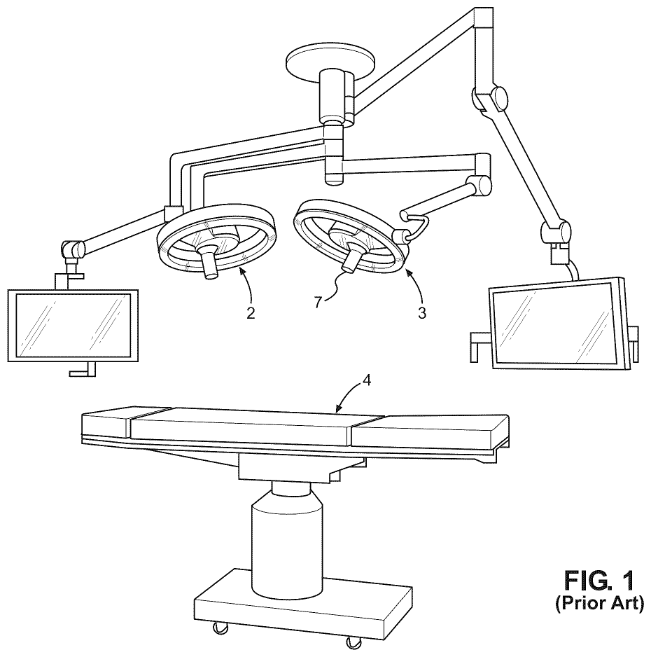

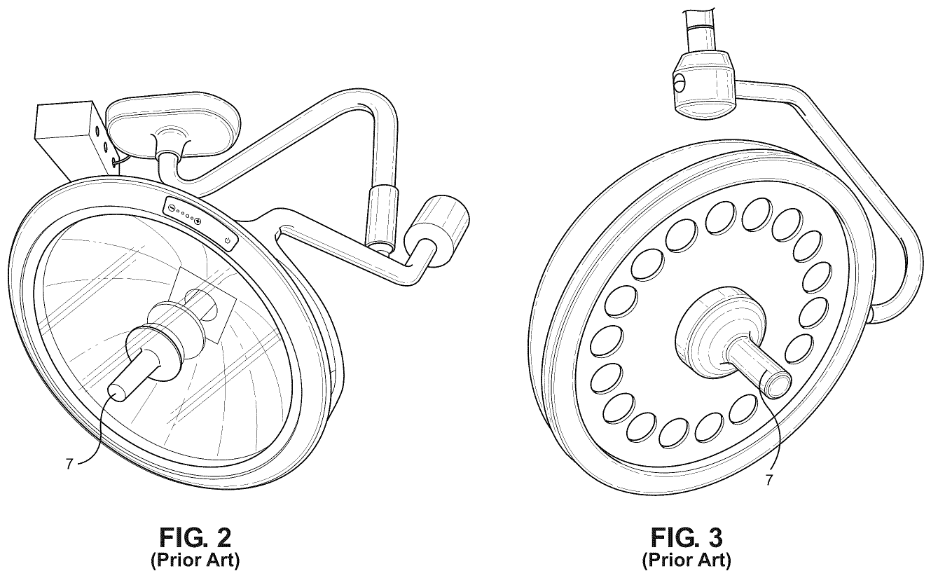

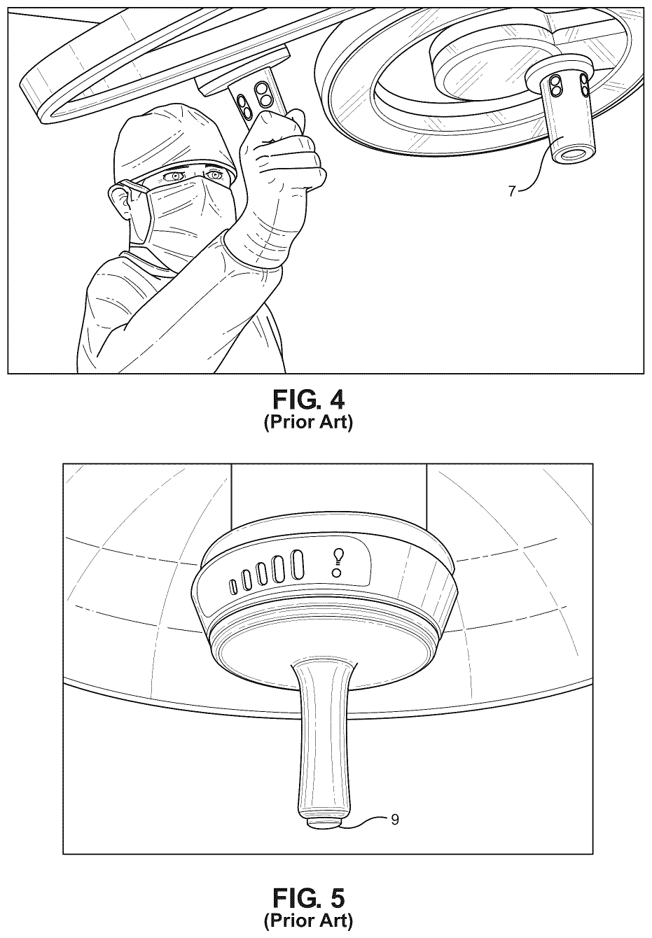

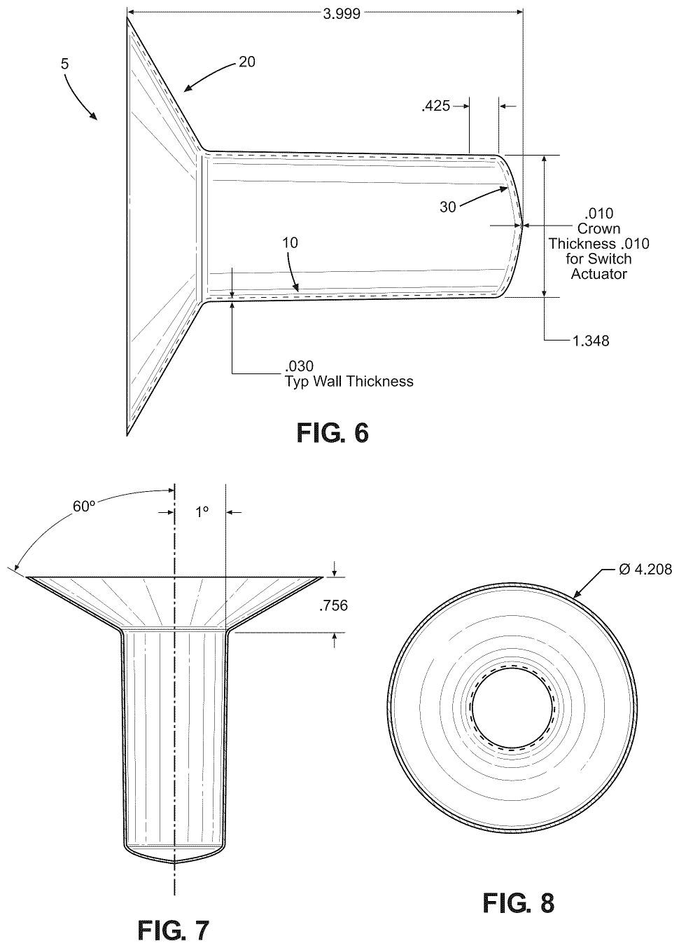

Disclosed herein is a cover 5 that readily slides onto most or all of the different types of handles found on conventional operating room lights that are commonly used in operating rooms. Some examples of such operating room lights are shown in FIGS. 1-5. A side view in section of one embodiment of the disclosed cover is shown in FIG. 6 with measurements shown in inches. The disclosed cover has a substantially cylindrical portion 10 integral with a conical portion 20 that is tapered at the open end to allow placement of the cover 5 on the similarly-shaped light handle. The conical portion 20 guides the shaft into the hollow barrel interior of the substantially cylindrical portion 10.

As noted in FIG. 6, the thickness of the sidewall of the cylindrical portion 10 may be about 0.030 inches. The diameter of the inner wall of the cylindrical portion 10 may be about 1.348 inches. The total length of the cover 5 may be about 3.999 inches. The thickness of the crown at the end 30 of the cylindrical portion may be about 0.010 inches. The crown is the location where the switch actuator may be, and so having a sidewall thickness that is in this range may more readily permit actuation of the switch by a human operator. The diameter of the conical portion 20 or base may be about 4.208 inches.

A nurse or other installer will typically push the substantially cylindrical portion 10 onto the light handle shaft and possibly give it a tap with his/her hand to seat it. When the cover 5 is pushed so the distal end of the light handle's shaft reaches the end 30, the substantially cylindrical portion 10 fits tightly to the shaft of the light handle. The cover 5 is thinner in sidewall thickness at the end 30 where some handles have switches so that a person can push the mechanical button up and down to adjust the light, even when the cover 5 is on the handle 7.

As shown in FIG. 7, the sidewall of the cylindrical portion 10 is tapered by about one degree (this can be varied) so that the hollow barrel of the cover 5 near the closed end 30 of the substantially cylindrical portion 10 is slightly narrower to grip the light handle's shaft near the end where a light switch may be. The material of which the cover is preferably made is similar to a silicone material, meaning that it is highly elastic and highly flexible. An elastomer is one acceptable material. A human user can readily stretch the cover's sidewall to fit the cover 5 on the light handle's shaft 7, even if the shaft 7 varies in shape or diameter along its length. This permits the cover 5 to be placed on a light handle 7 by the sidewall of the substantially cylindrical portion 10 stretching and gripping the shaft near the end 30.

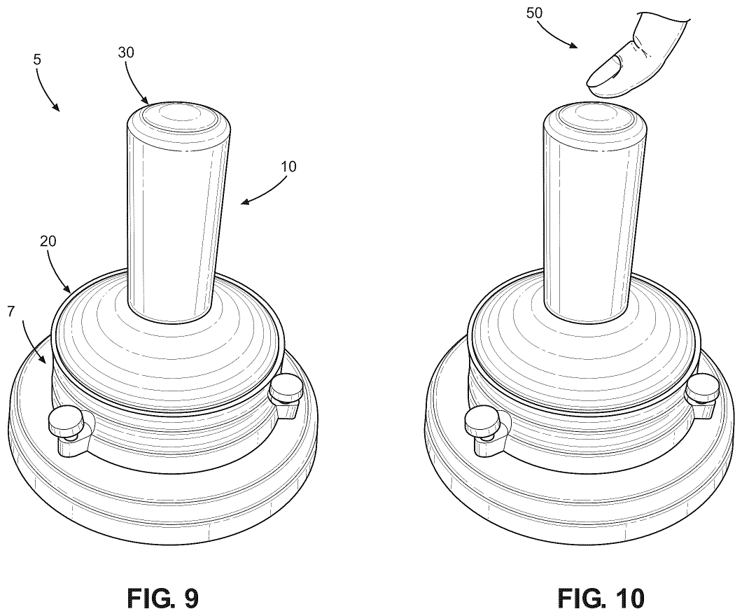

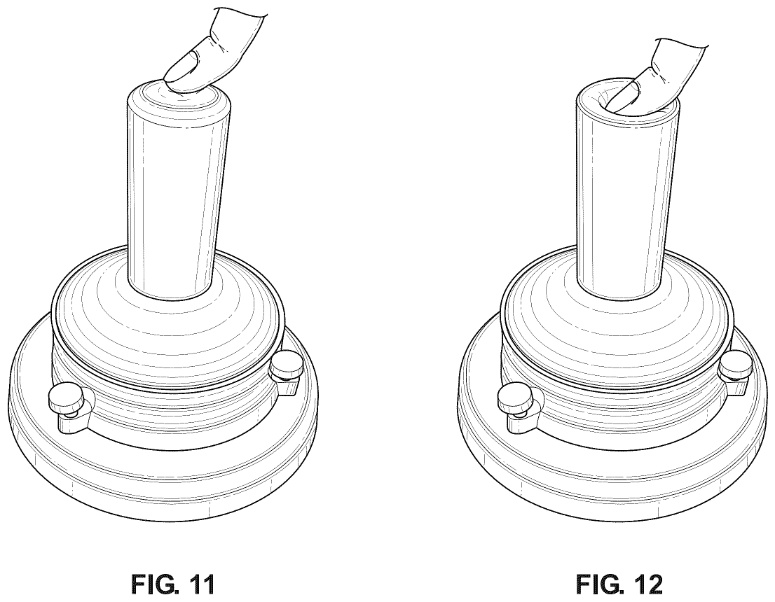

FIGS. 9-12 show the cover 5 combined with a conventional light handle 7. The actuation of the light switch (located at the end of the light handle) through the end 30 of the cover 5 is shown in sequence in FIGS. 9-12, and described below, after the cover 5 is combined with a conventional light handle 7. The cover 5 is shown in FIG. 9 in an operable position on a conventional handle 7 prior to actuation of the light switch. Actuation begins in FIG. 10 with an instrument or a human finger 50 approaching the end 30. The finger 50 makes contact with the end 30 in FIG. 11, and continues in FIG. 12 moving toward the switch. The flexible material of which the end 30 is constructed permits the user to simply deform the end 30 (by flexion and stretching of the cover's 5 sidewall) into the switch. The finger 50 depresses the switch in a conventional manner, almost as if the end 30, which is very flexible and elastic, were not present. Nevertheless, the end 30 is present and prevents any microbe or other transmission to the light switch. Upon release of the finger 50 or instrument, the end 30 returns to its original shape as shown in FIGS. 9-11, and the finger 50 is withdrawn. If the switch is to be actuated again, the process is repeated.

An embodiment of the invention is the cover 5 having the specific features described above. Another embodiment of the invention is the combination of the cover 5 and an operating room light handle 7, as shown in the figures and described above, as well as known conventionally. The combination of the cover 5 and the light handle is shown in FIGS. 9-12.

This detailed description in connection with the drawings is intended principally as a description of the presently preferred embodiments of the invention, and is not intended to represent the only form in which the present invention may be constructed or utilized. The description sets forth the designs, functions, means, and methods of implementing the invention in connection with the illustrated embodiments. It is to be understood, however, that the same or equivalent functions and features may be accomplished by different embodiments that are also intended to be encompassed within the spirit and scope of the invention and that various modifications may be adopted without departing from the invention or scope of the following claims.

1. A cover for a light handle, the cover comprising:
(a) a substantially cylindrical portion having a barrel defined by a sidewall;
(b) a conical portion disposed near a first end of the cylindrical portion, at least a portion of the conical portion having a larger diameter than the cylindrical portion; and
(c) an end portion disposed near a second end of the cylindrical portion opposite the conical portion.

2. The cover for a light handle in accordance with claim 1, wherein at least the substantially cylindrical portion and the end portion are made of an elastomeric material.

3. The cover for a light handle in accordance with claim 1, wherein the substantially cylindrical portion is tapered so that the first end has a larger diameter than the second end.

4. The cover for a light handle in accordance with claim 1, wherein a sidewall thickness of the cylindrical portion is larger than a sidewall thickness of the end portion.

5. A combination of a cover and a light handle, the combination comprising:
(a) a substantially cylindrical portion having a barrel defined by a sidewall in which the light handle is disposed;
(b) a conical portion disposed near a first end of the cylindrical portion over a base of the light handle, at least a portion of the conical portion having a larger diameter than the cylindrical portion; and
(c) an end portion disposed near a second end of the cylindrical portion opposite the conical portion and adjacent a light switch of the light handle.

6. The combination in accordance with claim 5, wherein at least the substantially cylindrical portion and the end portion are made of an elastomeric material.

7. The combination in accordance with claim 5, wherein the substantially cylindrical portion is tapered so that the first end has a larger diameter than the second end.

8. The combination in accordance with claim 5, wherein a sidewall thickness of the cylindrical portion is larger than a sidewall thickness of the end portion.
